(19) United States
(12) Patent Application Publication
(10) Pub. No.: US 2011/0092764 A1
(43) Pub. Date: Apr. 21, 2011

(54) Harrison human male permanent penile extender device (HHMPPPE)

(76) Inventor: Wilbur E. Harrison, JR., Melbourne, FL (US)

(21) Appl. No.: 12/589,096

(22) Filed: Oct. 19, 2009

(51) Int. Cl. A61F 5/00

(52) U.S. Cl. 600/40

(57) ABSTRACT

The Surgically Installed Harrison Human Male Permanent Penile Erection and Extension Device (HHMPEED) is shown schematically in FIGS. 1, 2, 3, and 4 of this patent application. The (HHMPEED) consists of two (2) basic front and rear extender units as shown, plus three (3) optional extension units which allow optional lengthening of the penis by some four (4) inches or more [up to some 12 inches or more], depending upon the capability to be stretched or extended by the penis nerves [particularly the nerves controlling the penis organism and ejaculation], as well as the blood arteries and veins to also be stretched or extended. The primary objective of the HHMPSED is to help married couples, who may be having difficulty impregnating the wife for the purpose of having a baby or perhaps multiple babies. Please note: personal safety considerations, as well as personal functional considerations positively require that the (HHMPEED) be surgically installed by a highly trained, experienced and fully competent professional Urologist or Surgeon.

### BACKGROUND OF THE INVENTION

1. Field of the Invention

The Harrison Human Male Permanent Penile Extension Device (HHMPPEED) is drawn schematically in FIGS. 1, 2, 3 and 4 of this patent application. The (HHMPPEED) consists of two (2) basic front and rear extender units as shown, plus three (3) optional extension units which allow optional lengthening of the penis by some four (4) inches or more [up to some 12 inches or more], depending upon the capability to be stretched or extended of the penis nerves [particularly the nerves controlling the penis organism and ejaculation], as well as the blood arteries and veins to also be stretched or extended (see FIG. 3 and FIG. 4). The primary objective of the HHMPPEED is to help married couples, who may be having difficulty impregnating the wife for the purpose of having a baby or perhaps having multiple babies. Please note: personal safety considerations, as well as personal functional considerations positively require that the (HHMPPEED) be surgically installed by a highly trained, experienced and fully competent professional Urologist or Surgeon.

2. Description of the Prior Art

There is known prior art for penis stiffening devices that have pointed ends that enable insertion into a human male penis to be easier for the surgeon installing such devices. However, such devices do not allow for the length extension of the human male penis because a pointed device would “migrate” through the head of the penis by a process known as “cold flow” in metals, or “migration” as happens in a human body with a bullet wound. This is why a bullet must be removed, if at all possible, from a human body, because otherwise, the bullet will move (migrate) through the human body and cause secondary, sometimes fatal, problems. The (HHMPPEED) bulbous ends eliminate the “migration” problem by normalizing stress distribution on the penis head, thereby allowing the penis to be stretched or extended. This penis extension is limited by the distance that the Dorsal Nerve, Dorsal Artery and other nerves that control or influence the human male ejaculation process can be stretched or extended.

### SUMMARY OF THE INVENTION

Accordingly, the current invention, which shall be described subsequently in greater detail, is offered to attain the above objectives. Representative embodiments of the concepts of the current invention are illustrated in the drawings of FIG. 1 of 2 and FIG. 2 of 2.

### BRIEF DESCRIPTION OF THE DRAWINGS

FIGS. 1 and 2 are a schematic drawing of the surgically installed Harrison Human Male Permanent Penile Erection and Extension Device (HHMPPEE).

FIGS. 3 and 4 are both depictions of cross sections of a typical human male reproductive organ.

### DESCRIPTION OF THE PREFERRED EMBODIMENT

With reference now to the Figures, the following shall be described:

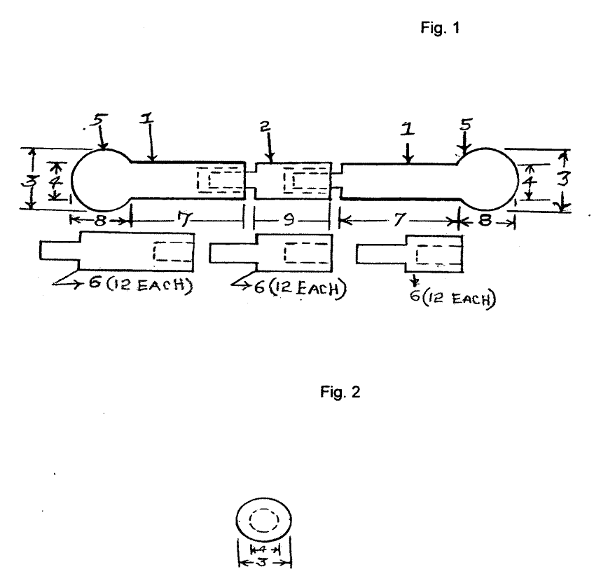

FIG. 1: More specifically, it is noted herewith that the first embodiment of FIGS. 1 and 2 includes [Scale: 10 mm (millimeters)=0.3937 inches)]

1 denotes the 10 millimeter (mm)/(0.3937 inch) diameter and the 30 mm (1.1811 inch) long human male penis extender front and rear sections which are each bulbous on one end.

2 denotes the 10 millimeter (mm) (0.3937 inch) diameter, and the 20 mm (0.7874 inch) long middle extension unit.

3 denotes the 17.5 mm diameter (0.688975 inch bulbous front and rear sections of the device.

4 denotes the 10 mm (0.3937 inch) diameter of the extender units, not including the bulbous front and rear sections, which are 17.5 mm (0.688975 inch) in diameter.

5 denotes the bulbous front and rear sections of the device.

6 denotes the extension which are included a several sets with each device.

7 denotes the 30 mm (1.1811 inch) length of the extension units, not counting the length of the bulbous ends.

8 denotes the 17 mm (0.66929 inch) ends of the extension units.

9 denotes the 20 mm (0.7874 inch) length of the middle of one of the three extension base units.

The (HHMPPEE) shall be constructed of solid Teflon, white silicone rubber or another material that is not rejected by the human body.

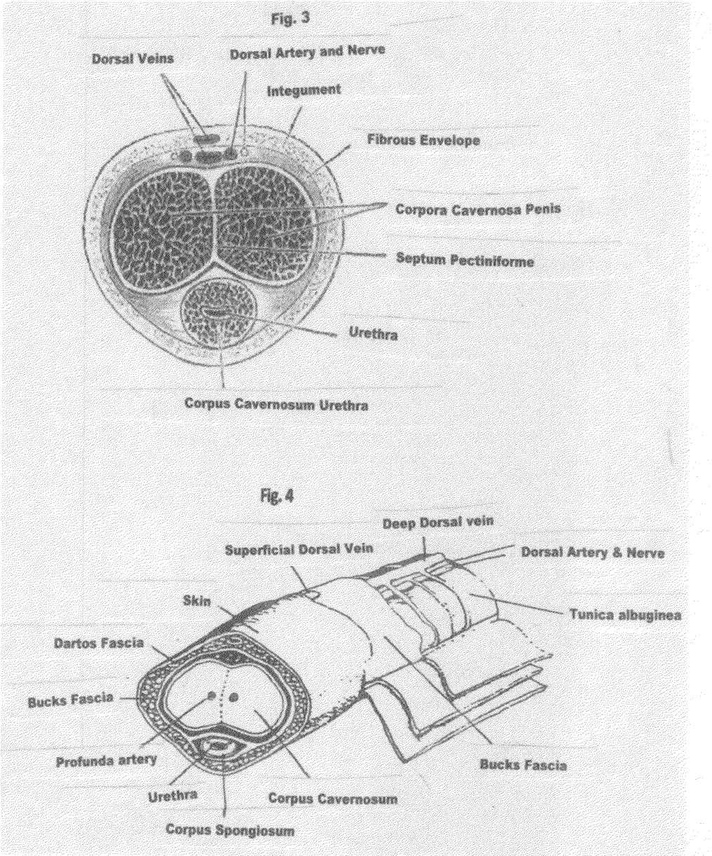

FIGS. 3 and 4: More specifically, it is noted herewith that the first embodiment of FIGS. 3 and 4 shows: two sectional drawings of the human male reproduction system. Please note the location of the Dorsal Nerve, the Dorsal Artery, the Dorsal Veins, etc. which must be stretched or extended when the human male penis is extended in length. Also shown is the two Corpora Cavernosa of the penis which is the normal/natural penis extender and stiffener required for penetration of the human female vagina. The (HHMPPEE) is surgically inserted into these two spaces.

This surgical procedure is not considered to be complex or life threatening, as long as the male is physically able to be anesthetized (which is normally a local anesthetic).

### SPECIFICATION

1. The surgically installed Harrison Human Male Permanent Penile Erection and Extension Device (HHMPEED), as diagramed schematically in FIG. 1 and FIG. 2 of this patent application, is configured with bulbous front and rear pieces of the (HHMPEED) which allows the permanent erection and extension of the human male penis by some 4 to 10 inches, or more, beyond the natural or normal erection length. This allows penetration of the human female vagina. This extension is mutually determined by the request of the human male patient, the medical installation professional and the effective stretch or extension distance that the Dorsal Nerve, Dorsal Artery and other physical/medical factors allow.

2. The extendable feature of the multiple interlocking extender sections that are provided with each (HHMPEED) provide for the extension of the human male penis, as mutually determined by the request of the human male patient, the medical installation professional and the effective stretch or extension distance that the Dorsal Nerve, Dorsal Artery and other physical/medical factors allow.

3. The (HHMPEED) shall be constructed of pure Teflon, White Silicone Rubber or another material that shall not be rejected by the human body.

1. The surgically installed Harrison Human Male Permanent Penile Erection and Extension Device (HHMPPEED), as diagramed schematically in FIG. 1 of 2 of this patent application, is configured with the bulbous front and rear pieces of the (HHMPPEED), plus the multiple interlocking extender sections that are provided with each (HHMPPEED), enables/allows the permanent erection (stiffening) and extension of the human male penis by some 4 to 10 inches, or more, beyond the natural or normal erection length. This allows the penetration of the human female vagina when mutually agreed to by the human male and the human female. The extent of this extension is mutually determined by the request of the human male patient, the medical installation professional and the effective stretch or [extension] distance that the Dorsal Nerve, Dorsal Artery and other physical medical factors allow. This is the first unique patentable feature claimed by this patent application.

2. The second claim here is that the extension of the male penis, as described in 1 above, shall materially improve the chances of impregnation of the human female with 1 baby or multiple babies.
